(19) United States
(12) Patent Application Publication
(10) Pub. No.: US 2026/0315673 A1
(43) Pub. Date: Oct. 15, 2026

(54) DETERMINATION OF EXHAUST GAS FLOW OF GAS EXCHANGE DEVICE

(71) Applicant: Maquet Cardiopulmonary GmbH, Rastatt (DE)

(72) Inventors: Christian LODERER, Rastatt (DE); Dimitri PROBST, Rastatt (DE)

(21) Appl. No.: 19/684,873

(22) Filed: May 21, 2026

Related U.S. Application Data
(63) Continuation of application No. PCT/EP2024/083039, filed on Nov. 20, 2024.

(30) Foreign Application Priority Data
Nov. 23, 2023 (SE) 2351343-5

(51) Int. Cl. A61M 1/16

(52) U.S. Cl. CPC A61M 1/1698; A61M 2205/3334; A61M 2230/205

(57) ABSTRACT

A method for determining a total flow rate of exhaust gas exiting a gas exchange device coupled to a circulatory system of a patient is disclosed. The gas exchange device is configured to provide oxygen-rich blood to the circulatory system by exposing oxygen-poor blood to oxygen provided by an inlet gas and to allow passage of excess oxygen to the exhaust gas. The method involves determining the total flow rate of exhaust gas from a concentration and flow rate of oxygen leaving the gas exchange device. The concentration is retrieved from a sensor, whereas the flow rate is determined based on a mass equilibrium approach, in which the amount of oxygen leaving the gas exchange device corresponds to a difference between the amount of oxygen supplied to the gas exchange device and the amount of oxygen taken up by the blood during the gas exchange.

### CROSS-REFERENCE TO RELATED APPLICATIONS

This application is a continuation under 35 U.S.C. § 120 of International Application No. PCT/EP2024/083039, filed Nov. 20, 2024 which claims priority to Swedish Application No. SE 2351343-5, filed Nov. 23, 2023, under 35 U.S.C. § 119 (a). Each of the above-referenced patent applications is incorporated by reference in its entirety.

### TECHNICAL FIELD

The present disclosure relates to methods and systems for gas exchange in a circulatory system of a patient, using a gas exchange device. More particularly, the present disclosure concerns techniques for determining a flow rate of exhaust gas exiting such a gas exchange device.

### BACKGROUND

Gas exchange in blood is a physical process by which gases move through a membrane, such as the blood-air barrier in the alveoli of a mammal lung, to allow oxygen to be taken up by the blood and carbon dioxide to be released from the blood.

In cases of respiratory failure or bypass, an external gas exchange device may be used to support or replace the function of a patient's lungs. The gas exchange device typically uses a sweep gas to oxygenate the blood and to allow carbon dioxide to be released from the blood.

The performance of the gas exchange device is crucial for patient safety and effective medical treatment. Too low uptake of oxygen or too low release of carbon dioxide may quickly lead to dangerous situations in which the health and safety of the patient is put at risk. A thorough monitoring of the flow rate and composition of the sweep gas is therefore desirable.

One commonly monitored parameter is the oxygen uptake in the blood, which may be estimated by a difference in oxygen content of the incoming sweep gas and the oxygen content of the outgoing sweep gas, i.e., the oxygen content before and after the gas exchange has taken place. This estimation typically requires the total flow rate of the sweep gas and the oxygen concentration to be known.

While the incoming flow rate may be measured directly with an appropriate measuring device, it is more challenging to determine the outgoing flow rate with high accuracy. A direct flow rate measurement at an outlet of the gas exchange device risks missing leakages, such as sweep gas escaping through an emergency outlet which is commonly implemented in many gas exchange devices. Further, the outgoing sweep gas tends to be saturated with water vapour, which may condensate and thereby limit the accuracy of the flow rate measurements.

Therefore, there is a need for improved and alternative techniques for monitoring the flow rate of gas passing through a gas exchange device in general, and in particular to determine a total flow rate of exhaust gas exiting such a gas exchange device.

### SUMMARY

In view of the above, the present disclosure provides an improved or alternative technology having the features set out in the independent claims.

Hence, according to a first aspect, there is provided a method for determining a total flow of exhaust gas exiting a gas exchange device coupled to a circulatory system of a patient, wherein the gas exchange device is configured to provide oxygen-rich blood to the circulatory system by exposing oxygen-poor blood of the patient to oxygen provided by an inlet gas and to allow passage of excess oxygen to the exhaust gas. The method comprises:
receiving sensor data indicating an oxygen content in the oxygen-rich blood and an oxygen content in the oxygen-poor blood;
determining an oxygen uptake in the blood based at least in part on a flow rate of blood through the gas exchange device and a difference between the oxygen content in the oxygen-rich blood and the oxygen content in the oxygen-poor blood;
determining a flow rate of oxygen in the inlet gas based at least in part on an oxygen concentration in the inlet gas and a total flow rate of the inlet gas;
determining a flow rate of oxygen in the exhaust gas based at least in part on a difference between the flow rate of oxygen in the inlet gas and the oxygen uptake in the blood;
receiving sensor data indicating an oxygen concentration in the exhaust gas; and
determining the total flow rate of the exhaust gas based at least in part on the flow rate of oxygen in the exhaust gas and the oxygen concentration in the exhaust gas.

In a second aspect, a system for exchanging one or more gases with blood in a circulatory system of a patient is provided. The system comprises a gas exchange device having a gas inlet for receiving an inlet gas and an outlet for expelling an exhaust gas, wherein the gas exchange device is configured to provide oxygen-rich blood by exposing oxygen-poor blood of the patient to oxygen provided by the inlet gas and to allow passage of excess oxygen to an exhaust gas. The system also comprises a sensor arrangement configured to generate sensor data indicating an oxygen content in the oxygen-rich blood, an oxygen content in the oxygen-poor blood, and an oxygen concentration in the exhaust gas. Further, the system comprises one or more processors and one or more non-transitory computer-readable media storing instructions executable by the one or more processors, wherein the instructions, when executed, cause the system to perform operations comprising:
determining an oxygen uptake in the blood based at least in part on a flow rate of blood through the gas exchange device and a difference between the oxygen content in the oxygen-rich blood and the oxygen content in the oxygen-poor blood;
determining a flow rate of oxygen in the inlet gas based at least in part on an oxygen concentration in the inlet gas and a total flow rate of the inlet gas;
determining a flow rate of oxygen in the exhaust gas based at least in part on a difference between the flow rate of oxygen in the inlet gas and the oxygen uptake in the blood; and
determining the total flow rate of the exhaust gas based at least in part on the flow rate of oxygen in the exhaust gas and the oxygen concentration in the exhaust gas.

In the above aspects, the total flow rate of exhaust gas is obtained from the concentration and flow rate of oxygen leaving the gas exchange device. The concentration may be retrieved from a sensor, whereas the flow rate may be determined based on a mass equilibrium approach in which the amount of oxygen leaving the gas exchange device is assumed to correspond to the difference between the amount of oxygen supplied to the gas exchange device and the amount of oxygen taken up by the blood during the gas exchange. The amount of oxygen taken up by the blood may be determined by comparing the oxygen content in the oxygen-rich blood with the oxygen content in the oxygen-poor blood based on blood sensor data. By subtracting the amount of oxygen that is taken up by the blood from the amount of oxygen that is supplied to the gas exchange device, it is possible to determine the amount of oxygen that remains in the sweep gas after the gas exchange. By combining the amount of oxygen in the exhaust gas with the concentration of oxygen in the exhaust gas, the total flow rate of exhaust gas can be determined without directly measuring the flow rate using, for example, a flow meter.

Direct measurements of the total flow rate of exhaust gas may be challenging for several reasons. Firstly, the exhaust gas may be saturated with water vapour and may have an elevated temperature corresponding to, or being close to, the body temperature of the patient. This is associated with a considerable risk for the water vapour condensing, which in turn may reduce the accuracy of the flow rate measurements, as condensed water may interfere with the sensor readings, block pathways of the sensor, and effectively change the relative concentration of other gases, including oxygen. Secondly, many gas exchange devices are for patient safety reasons provided with an additional gas outlet, which often is arranged in a constant open state to allow a portion of the exhaust gas flow to pass therethrough. The additional gas outlet forms an emergency outlet that prevents overpressure to form in the gas exchange device, should the primary outlet for some reason be obstructed. As a portion of the exhaust gas may escape through the emergency outlet instead of the primary outlet, a flow sensor arranged at the primary outlet would risk providing an inaccurate measure of the total flow rate of exhaust gas. Approximating the total flow rate of exhaust gas with the flow rate of inlet gas, as in some prior art technologies, risks being particularly inaccurate for gas exchange devices where there is a leakage, such as through an emergency outlet.

The present aspects hence provide an alternative to methods in which the total flow rate of exhaust gas is determined by direct flow rate measurements or is approximated by the flow rate of inlet gas.

In the context of the present disclosure, the term “flow” or “flow rate” typically refers to how the gas is moved through the system. The flow rate may either refer to the total flow rate of gas supplied to the gas exchange device or exhausted by the gas exchange device, or the flow rate of a particular component of the gas, such as oxygen or carbon dioxide. The flow rate may generally be understood as a term describing the quantity of a gas moving through a given cross-sectional area per unit time and can be measured in terms of either volume or mass. The former may be referred to as a volumetric flow rate and describes how much volume of a gas passes through a given area in a specific time. In the SI system, the volumetric flow rate is often expressed in cubic metres per second (m³/s). However, in the present context the flow rate may commonly be expressed in litres per minute (L/min). Thus, the volumetric flow rate typically describes how many litres of gas, such as oxygen, are being supplied to (or exhausted by) the gas exchange device per minute. Alternatively, the flow rate may be measured as a mass flow rate, describing the mass of a gas (such as oxygen) passing through a given area in a specific time. The mass flow rate may thus be expressed in kilograms per second (kg/s). Mass flow rate may be particularly useful when the density of the gas might change under varying conditions, as the mass flow rate may provide a measure that is independent of temperature, pressure, and moisture content. It will be appreciated the volumetric flow rate is proportional to the mass flow rate, and that the volumetric flow rate therefore be converted into a mass flow rate (and vice versa) for a given temperature and pressure of the gas.

The standard cubic centimetre per minute (SCCM) is an example of a unit that can be used to quantify the flow rate of a fluid and may be understood as the mass flow rate of one cubic centimetre per minute of a gas at a density defined at some standard conditions for temperature and pressure. The standard conditions may vary between different regulatory bodies but may in an example correspond to a temperature of 0° C. and a pressure of 1.01 bar.

The oxygen content in the blood typically refers to the total concentration of oxygen carried by the blood flow. Oxygen typically exists in the blood in two major forms: bound to haemoglobin and dissolved directly in the plasma. It will be appreciated that other forms may exist, but that these two tend to account for most of the oxygen content. Commonly, the major part of the oxygen transported in the blood is bound to haemoglobin, whereas a smaller fraction of the oxygen in the blood is dissolved in the plasma. Hence, the oxygen content typically refers to the total concentration of oxygen carried by haemoglobin and by the plasma. The oxygen content may be expressed as a volume of oxygen per unit volume of blood (such as millilitres of oxygen per decilitre of blood) or amount of substance of oxygen per unit volume of blood (such as millimoles of oxygen per decilitre of blood).

The oxygen concentration in the inlet gas and the exhaust gas may typically be measured as a percentage of the total gas mixture. For example, medical oxygen supplied to the gas exchange device may have concentrations from around 21% (corresponding to the concentration in air) to 100%, depending on the clinical need. The percentage typically describes a volume ratio, like litres of oxygen per litre of total gas. However, in some examples the concentration may also be expressed as a mole fraction (number of moles of oxygen per mole of gas mixture) or an amount of substance oxygen per unit volume of gas mixture.

Oxygen-poor blood typically refers to blood that has circulated through the body and has delivered oxygen to the tissues. As a result, it may have a higher concentration of carbon dioxide. The oxygen-poor blood may also be referred to as deoxygenated blood or venous blood. The oxygen-poor blood may be supplied to the gas exchange device for enrichment with oxygen.

Oxygen-rich blood, also referred to as oxygenated blood or arterial blood, is understood as blood that has been enriched with oxygen by gas exchange device. The oxygen-rich blood may be returned to the patient's body to supply the tissues with oxygen.

As mentioned above, the oxygen content in the blood may refer to the total concentration of oxygen carried by the blood flow, including both oxygen bound to haemoglobin and oxygen dissolved in the plasma. Thus, the sensor data may indicate both—i.e., the concentration of oxygen bound to haemoglobin in the oxygen-rich blood and in the oxygen-poor blood, as well as the concentration of oxygen dissolved in the blood plasma of the oxygen rich-blood and in the oxygen-poor blood. By taking both the concentration of oxygen bound to haemoglobin and the concentration of oxygen dissolved in plasma into account, the total concentration or amount of oxygen taken up by the blood during the gas exchange may be determined at a relatively high accuracy.

Alternatively, or additionally, for oxygen-poor blood, or blood which is not fully saturated with oxygen, the concentration of oxygen bound to haemoglobin can be determined based on the concentration of oxygen dissolved in plasma. This is based on the establishment of a known relationship between the concentration of oxygen dissolved in plasma (also referred to as partial pressure of oxygen) and the concentration of oxygen bound to haemoglobin (also referred to as the oxygen saturation of haemoglobin). Thus, by measuring the partial pressure of oxygen in the oxygen-poor blood, the relationship may be used to estimate the oxygen saturation without directly measuring the oxygen saturation level.

Various sensors may be employed to measure the oxygen concentration in the exhaust gas. However, condensation of water vapour in the exhaust gas has been observed to pose challenges, as it may interfere with the sensor and affect the relative concentration of other gases, including oxygen. This may in some examples be addressed by reducing the temperature of the exhaust gas to allow water vapour to condensate in a more controlled way before the sensor is exposed to the gas. The exhaust gas may be cooled to ambient room temperature or to even lower temperatures to further reduce the humidity of the exhaust gas. In further examples, the sensor may be heated directly (direct on or adjacent the sensor chip) or indirectly (whole sensor assembly/tube) to reduce the risk of condensed water interfering with the operation of the sensor. Additionally, or alternatively, the exhaust gas may be brought to pass through a device allowing water vapour to be removed from the gas. An example of such as device is the Nafion tube, which will be described in further detail with reference to FIG. 1. Combinations are also possible, in which the exhaust gas is cooled, and the sensor heated to further reduce the risk of condensation.

It will be appreciated that the sensor for measuring the oxygen concentration in the exhaust gas may be integrated in the gas exchange device, i.e., permanently or replaceably installed at, for example, an outlet of the gas exchange device. However, in other examples, the sensor may be a separate unit configured to measure the exhaust gas downstream the gas exchange device.

In some examples, a flow of carbon dioxide in the exhaust gas may be determined based on the total flow rate of the exhaust gas as well as sensor data indicating a carbon dioxide concentration in the exhaust gas. The flow rate of carbon dioxide may be used as an indication of a performance of the gas exchange device, and more specifically the efficiency with which the gas exchange is performed. Relatively low, or decreasing, concentrations or flow rates of carbon dioxide in the exhaust gas may indicate that a membrane of the gas exchange device, through which the gas exchange between the sweep gas and the blood takes place, is operating at a reduced capacity. Common reasons may be clogging or blockage of the membrane, as well as deposition on the membrane fibres, which may increase the diffusion resistance and reduce the gas exchange efficiency. Hence, by comparing the concentration or flow rate of carbon dioxide in the exhaust gas with a threshold measure, a state of the gas exchange device may be determined. Should the concentration or flow rate of carbon dioxide meet or exceed the threshold measure, the gas exchange device may be determined to perform as intended. Should the concentration or flow rate of carbon dioxide be lower than the threshold measure, a warning or signal indicating that the gas exchange device needs to be replaced, may be issued. The threshold measure rate may be a predetermined reference value based on, for example, an operating point of the gas exchange device defined by the inlet flow rate of sweep gas, the flow rate of blood through the gas exchange device, and the amount of carbon dioxide dissolved in the blood.

In the above examples, some parameters for determining the total flow of exhaust gas may be predetermined or known from system settings, whereas other parameters may be retrieved from sensor data. The flow of blood through the gas exchange device, that is, the flow rate of blood with which the gas exchange may take place, may be measured by means of an ultrasonic flow sensor, or be a known parameter determined by the settings of the system, such as the pump rate of a pump used for circulating the blood through the system. Further, the oxygen concentration in the inlet gas as well as the total flow of the inlet gas may be known from the settings of the inlet gas supply, such as a gas blender used to supply the gas exchange device with the required sweep gas. The oxygen content in the oxygen-rich blood and the oxygen-poor blood, as well as the oxygen concentration in the exhaust gas, may however be retrieved from sensor data.

The second aspect may generally have the same features and advantages as the first aspect. It is further noted that the disclosure relates to all possible combinations of features, unless explicitly stated otherwise.

### BRIEF DESCRIPTION OF THE DRAWINGS

The above, as well as additional object, features, and advantages of the present disclosure will be better understood through the following illustrative and non-limiting detailed description of examples of the present disclosure, with reference to the appended drawings, where the same reference numerals will be used for similar elements, wherein:

FIG. 1 shows a system according to an example, comprising a gas exchange device coupled to an extracorporeal circuit.

FIG. 2 shows examples of methods for determining a total flow of exhaust gas expelled by a gas exchange device.

As illustrated in the figures, the sizes of the elements and features may be exaggerated for illustrative purposes and, thus, are provided to illustrate the general structures of the examples. Like reference numerals refer to like elements throughout.

### DETAILED DESCRIPTION

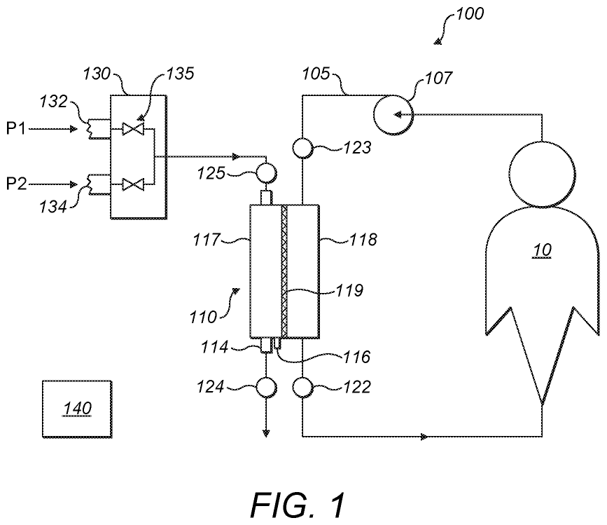

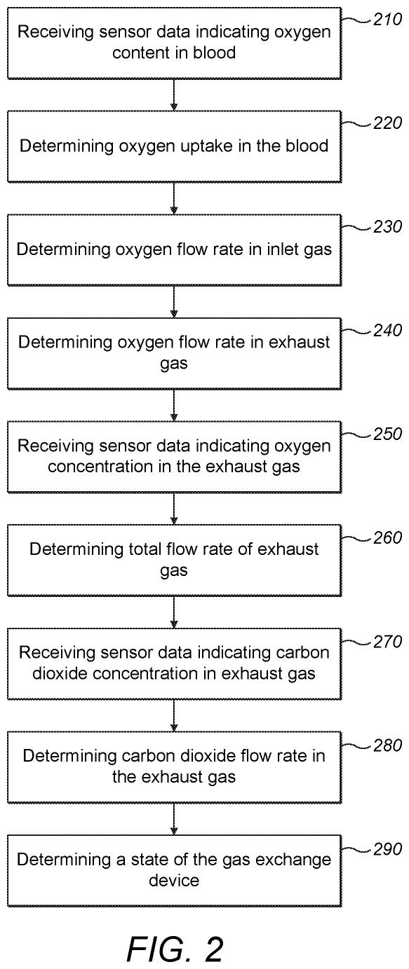

FIG. 1 shows by way of example a system 100 for exchanging one or more gases with blood in a circulatory system of a patient 10. The system 100 comprises a gas exchange device 110 configured to provide oxygen-rich blood by exposing oxygen-poor blood of the patient 10 to oxygen provided by an inlet gas and to allow passage of excess oxygen to an exhaust gas. The system further comprises a sensor arrangement 122, 123, 124 configured to generate sensor data indicating an oxygen content in the oxygen-rich blood, an oxygen content in the oxygen-poor blood, and an oxygen content in the exhaust gas. The sensor data may be processed by a processor 140 configured to determine a total flow rate of the exhaust gas based on the sensor data, as will be described in further detail in connection with FIG. 2.

Examples of systems 100 according to the present disclosure include cardiopulmonary bypass machines (heart-lung machine) providing circulatory and respiratory support in the operating theatre while the heart is stopped for surgery, extracorporeal membrane oxygenator (ECMO) devices providing circulatory and respiratory support in intensive care units, and extracorporeal carbon dioxide removal (ECCO2R) devices removing excess carbon dioxide from the blood.

In the present example, the gas exchange device 110 is arranged in an extracorporeal circuit 105 in which blood of the patient is circulated through the gas exchange device 110 by means of a circulation unit 107. The extracorporeal circuit 105 typically comprises fluid lines, or circuit tubing, for connecting the various components of the extracorporeal circuit and carrying the patient's blood through the gas exchange device 110.

The circulation unit 107 may, for example, comprise a roller pump or a centrifugal pump, depending on the specific type of system 100. Roller pumps typically comprises a rotating roller compressing a flexible tube or membrane. As the roller rotates, it squeezes the tube, creating a pulsatile flow of blood. Centrifugal pumps, on the other hand, use a rapidly rotating impeller to create a centrifugal force which pushes the blood outwards to generate a substantially continuous blood flow. Centrifugal pumps have been observed to reduce the risk for haemolysis, i.e., breakdown of red blood cells, and improve the patient comfort.

The gas exchange device 110 is configured to facilitate exchange of gases between the patient's blood and a sweep gas passing through the gas exchange device 110. Typically, the gas exchange device is configured to allow oxygen to pass from the sweep gas into the blood, thereby oxygenating the blood, and to allow carbon dioxide to be released from the blood and pass into the sweep gas. It will be appreciated that other types of gas and substances may be exchanged in a similar way, including anaesthetic agents and nitric oxide.

The gas exchange device 110, which in some examples may be referred to as an oxygenator, may comprise a gas region 117 and a blood region 118 separated by a gas-permeable membrane 119. The blood may be circulated through the blood region 118 by the circulation unit 107, whereas the sweep gas may flow through the gas region 117 via a gas inlet 112 and a gas outlet 114. At the gas inlet 112, the sweep gas may be referred to as an inlet gas, whereas the gas leaving the gas exchange device 110 may be referred to as an exhaust gas. Due to a partial pressure gradient, or a concentration difference, between individual components of the sweep gas and the corresponding component in the blood, this component may be passed through the membrane 119 from the gas region 117 into the blood region 118 or vice versa. In particular, oxygen may be passed from the gas region 117 into the blood region 118 through the membrane 119, so that the oxygen-poor blood is oxygenated into oxygen-rich blood. Vice versa, carbon dioxide may be passed from the blood region 118 into the gas region 117 through the membrane 119, so that carbon dioxide is removed from the flow of blood.

Several types of membranes 119 may be employed. In an example, the gas exchange device 110 comprises a hollow fibre membrane, in which the blood flows in the blood region 118 inside the fibres and the gas mixture flows in the gas region 117 outside the fibres. Oxygen and carbon dioxide exchange may occur across the membrane formed by the walls of the hollow fibres. In the example illustrated in FIG. 1, however, the membrane 119 is schematically illustrated as a sheet.

The transfer capacity of the oxygen or the carbon dioxide, i.e., the amount of carbon dioxide or oxygen which is supplied to or removed from the flow of blood per unit of time depends, inter alia, on the flow rate of the flow of blood, the flow of volume of the sweep gas, the composition of the sweep gas, and the state of the gas exchange device 110 itself. In particular, the gas transfer capacity of the membrane 119 can deteriorate over time due to accumulation of blood clots, deposition of a layer increasing diffusion resistance, and water vapour condensing inside, and blocking, the hollow fibres of the membrane. A gas exchange device 110 incapable of oxygenating the blood to a satisfying degree may hence be considered to be in a degraded or malfunction state.

The inlet gas may be provided by a gas blender 130. Typically, the gas blender 130 comprises a plurality of inlets 132, 134 configured to receive medical gas from a respective gas source (not shown). In the example shown in FIG. 1, the gas blender 130 comprises a first inlet 132 for a medical oxygen flow P1 and a second inlet 134 for a medical air flow P2. It will however be appreciated that more inlets may be provided, and that the gas blender 130 may be used for supplying other types of gas, such as nitric oxide or anaesthetic agents.

The medical oxygen may be supplied to the gas blender 130 from an oxygen source, which may be an oxygen outlet of a central medical gas system of a hospital facility, a pressurised gas cylinder, or any other type of oxygen source suitable for delivering oxygen for medical applications. The same applies to the air, which may be supplied from a pressurised air outlet of a central medical gas system of a hospital facility, a pressurised cylinder, or retrieved and pressurised from the surrounding air.

The gases received at the inlets 132, 134 may be mixed into a sweep gas mixture by a valve arrangement (collectively indicated by reference numeral 135) and supplied to the gas inlet 112 of the gas exchange device 110. The valve arrangement 135 may be configured to adjust the oxygen concentration and flow rate of the inlet gas according to the patient's needs, allowing the healthcare professionals to control the oxygen levels delivered to the patient. The valve arrangement 135 may be electronically controlled, and in some examples configured to utilise sensor input to achieve the accurate gas composition and flow rate.

The gas blender 130 may thus be operated to deliver a specific concentration of oxygen, such as 100% oxygen, at a specific flow rate, such as 4 litres per minute. The concentration and flow rate of oxygen may hence be known, or predetermined, by the operational settings of the gas blender 130, and used as input when calculating the total flow rate of the exhaust gas, as discussed below. Additionally, or alternatively, the mass flow rate of the gas may be determined. The mass flow rate may be determined either by means of a mass flow sensor, or by converting the volumetric flow rate into mass flow rate. The conversion typically requires the actual conditions, i.e., the temperature and the pressure, of the gas flow to be known, whereas the mass flow measurements typically require information pertaining to the composition of the gas flow to be known. For this purpose, a temperature sensor, a pressure sensor, and/or a mass flow sensor may be provided (collectively indicated by reference numeral 125).

In the example shown in FIG. 1, an emergency outlet 116 is provided at the gas region 117 of the gas exchange device 110. The emergency outlet 116 forms an additional outlet, i.e., in addition to the “primary” outlet 114 discussed above and serves to protect the gas exchange device 110 and the patient 10 from damage due to overpressure, should the primary outlet 114 be obstructed or blocked. The emergency outlet 116 may be arranged in a constant-open state, allowing exhaust gas to pass freely therethrough during operation of the gas exchange device 110. Hence, the total flow of exhaust gas may be divided in partial flows: a first partial flow passing through the gas outlet 114, and a second partial flow passing through the emergency outlet 116.

The gas exchange device 110 may be arranged to release the exhaust gas to the open air, to a gas capture device, or a gas evacuation arrangement (not shown in FIG. 1). The gas capture device, or gas evacuation arrangement, may be employed to recycle components of the exhaust gas or to prevent e.g. anaesthetic agents and hyperbaric oxygen to accumulate in the surrounding air. It will be appreciated that both the primary outlet 114 as well as the emergency outlet 116 may be coupled to such a capture device or evacuation arrangement.

One or more sensor arrangements may be provided to generate sensor data indicating an oxygen content in the oxygen-poor blood, an oxygen content in the oxygen-rich blood, and an oxygen content in the exhaust gas. In the example shown in FIG. 1, a first sensor arrangement 122 is arranged at a blood outlet of the gas exchange device 110 to measure the oxygen concentration in the oxygen-rich blood leaving the gas exchange device 110. Further, a second sensor arrangement 123 is arranged at a blood inlet of the gas exchange device 110 to measure the oxygen concentration in the oxygen-poor blood supplied to the gas exchange device 110.

The oxygen concentration of the blood (both upstream and downstream the gas exchange device 110) may refer both to oxygen bound to haemoglobin and oxygen dissolved in plasma. The amount of oxygen bound to haemoglobin may also be referred to as the oxygen saturation level and may be measured by means of optically based methods, in which the blood is illuminated, and the backscattered light measured to determine the saturation level. Light at two or more separate wavelengths may be used to further increase the accuracy of the measurements of the oxygen saturation level of the oxygen-rich blood and the oxygen-poor blood, respectively.

The amount of oxygen dissolved in plasma, which also may be referred to as the partial pressure of oxygen, may be measured using a blood gas sensor, typically an electrochemical sensor such as a Clark electrode, or an optical sensor such as an optode. The electrochemical sensor typically needs to be in contact with the blood, such as via a gas permeable membrane allowing the oxygen to pass towards a cathode, at which the oxygen is electrolytically reduced. The reduction reaction generates an electrical current which can be measured. The magnitude of this current is proportional to the concentration of dissolved oxygen in the blood.

Thus, in the example shown in FIG. 1, each of the first sensor arrangement 122 and the second sensor arrangement 123 comprises a sensor for measuring oxygen saturation, such as a photospectroscopic sensor, and a sensor for measuring partial pressure of oxygen, such as a Clark electrode or an optode. However, other sensor configurations and techniques are also possible, as will be readily known to a person of ordinary skill in the art. Further, in some examples, the oxygen saturation of the haemoglobin in the oxygen-rich and/or oxygen-poor blood may be estimated based on the partial pressure of oxygen, instead of being measured by a sensor. The estimation is based on the so-called oxygen dissociation curve, which is a known relation describing the affinity of haemoglobin for oxygen and how easily haemoglobin picks up and releases oxygen dissolved in plasma.

As shown in FIG. 1, the gas outlet 114 of the gas exchange device 110 may be provided with a sensor 124 for measuring an oxygen concentration of the exhaust gas, i.e., the gas expelled from the gas exchange device after the gas exchange process has taken place. The sensor 124 may, for example, be an electrochemical sensor producing a current proportional to the concentration of oxygen, a paramagnetic sensor measuring a change in a magnetic field caused by the presence of oxygen (due to the paramagnetic properties of oxygen), or an optical sensor measuring a phase shift of modulated light caused by oxygen molecules. Other types of sensors readily known to a person skilled in the art are also possible.

In some examples, a water vapour content in the exhaust gas may be reduced before the exhaust gas reaches the sensor 124 for measuring the oxygen concentration. By removing moisture from the exhaust gas, the risk for condensation is reduced, which in turn may improve the accuracy of the oxygen concentration measurements. The water vapour content may be reduced by cooling the gas, for example down to room temperature, and allowing water vapour to condense in a more controlled manner. Additionally, or alternatively, moisture may be removed by means of a Nafion tube (comprising a sulfonated tetrafluoroethylene-based fluoropolymer-copolymer), through which the exhaust gas may be brought to pass on its what to the sensor 124. A wall of the Nafion tube may typically be arranged to selectively permit water vapour to permeate through the tube wall, effectively “drying” the exhaust gas inside the tube. In further examples, the sensor 124 may be heated to reduce the risk for water vapour condensing on the sensor 124. The temperature of the sensor 124 may for example be increased by applying heat directly to the sensor chip, or by applying heat to a structure adjacent to, and in thermal contact with, the sensor 124.

Each of the sensors and sensor arrangements 122, 123, 124 discussed above may be an integrated part of the gas exchange device 110 (and thus be replaced together with the gas exchange device 110), or a separate element that can be attached to or into the gas exchange device 110. In the latter case, the sensor(s) may be reusable, i.e., used with more than one gas exchange device 110.

The sensors 122, 123, 124 may be configured to communicate with the processor 140 by means of a wired or wireless connection, such as a local-access network (LAN).

During operation of the system 100, oxygen-poor blood of the extracorporeal circuit is supplied to the blood region 118 of the gas exchange device 110, where it is oxygenated by the sweep gas flowing through the gas region 117 of the gas exchange device 110. The flow rate of blood passing through the gas exchange device 110 is in the following denoted Q_{blood}, the inlet flow rate of sweep gas is denoted Qᵢₙₗₑₜ, and the total exhaust flow rate of sweep gas leaving the gas exchange device 110 is denoted Qₑₓₕₐᵤₛₜ. The flow rate of blood Q_{blood} may typically ranges from 2 to 6 litres per minute (L/min), whereas each of the flow rate of inlet gas Qᵢₙₗₑₜ and the flow rate of exhaust gas Qₑₓₕₐᵤₛₜ typically ranges from 1 to 10 L/min.

The concentration, or fraction, of oxygen in the inlet gas is in the following denoted FᵢₙₗₑₜO₂, whereas the concentration of oxygen in the exhaust gas is denoted FₑₓₕₐᵤₛₜO₂. The concentration of oxygen in the sweep gas may typically be expressed as a volume ratio, i.e., litres of oxygen per litre of inlet/exhaust gas. Hence, the partial gas flow of oxygen (also referred to as flow rate of oxygen) in the inlet gas may be expressed as FᵢₙₗₑₜO₂·Qᵢₙₗₑₜ, and the flow rate of oxygen in the exhaust gas as FₑₓₕₐᵤₛₜO₂·Qₑₓₕₐᵤₛₜ. Typically, the inlet gas comprises 100% oxygen, whereby the flow rate of oxygen corresponds to the total flow rate of the inlet gas. After the gas exchange has taken place, the oxygen concentration in the exhaust gas (or “spent” sweep gas) may still be relatively high, such as about 95%. It will however be appreciated that the oxygen concentration in the exhaust gas may vary depending on several factors, such as the gas exchange efficiency between the sweep gas and the circulating blood, the flow rate of blood, the concentration of oxygen in the sweep gas, the saturation of oxygen in the oxygen-poor blood, and the concentration of haemoglobin.

As mentioned above, the flow rate of oxygen in the inlet gas may be predetermined by the operational settings controlling gas blender 130, whereas the flow rate of oxygen in the exhaust gas may be approximated by the flow rate of oxygen in the inlet gas minus any oxygen taken up by the blood during the gas exchange in the gas exchange device 110. Hence, by determining the flow rate of oxygen taken up by the blood and comparing it with the flow rate of oxygen supplied to the gas exchange device 110, the flow rate of oxygen expelled by the gas exchange device 110 can be estimated.

FIG. 2 is a flow chart of a method in which the flow rate of oxygen exiting the gas exchange device 110 is determined and used for calculating the total flow of exhaust gas expelled by the gas exchange device 110. The total flow of exhaust gas, in turn, may be used for determining the exchange rate of carbon dioxide, which is an indicator of the overall performance or state of the gas exchange device 110, as discussed below.

The gas exchange device 110 may be part of a system 100 similar to the one disclosed in FIG. 1 and may thus be coupled to the circulatory system of a patient to provide oxygen-rich blood by exposing oxygen-poor blood to oxygen in a sweep gas passing through a gas region 117 of the gas exchange device 110.

The method involves receiving 210 sensor data indicating an oxygen content in the oxygen-rich blood and an oxygen content in the oxygen-poor blood. The oxygen content may refer to the total concentration of oxygen that is bound to haemoglobin and oxygen that is dissolved in plasma. Typically, a major part of the oxygen is bound to haemoglobin, whereas a smaller fraction (such as around 1.5-3% of the total concentration of oxygen) is dissolved directly in the plasma. In the following, examples of how to determine the respective concentrations will be discussed with reference to FIGS. 1 and 2.

The concentration of oxygen bound to haemoglobin is proportional to a parameter referred to as oxygen saturation of the blood, which can be understood as the ratio of the available oxygen binding sites of the haemoglobin that are occupied by oxygen molecules. In a healthy adult individual, the oxygen saturation of oxygen-rich blood is usually between 95% and 100%, whereas the oxygen saturation of oxygen-poor blood is usually between 60% and 80%.

Haemoglobin is known to be able to carry about 1.34 mL of oxygen per gram when it is fully saturated. The concentration of haemoglobin in the blood varies between individuals and is preferably measured for each patient. Typically, the haemoglobin levels range from 13.5-18 g/dL in adult males, 12-15 g/dL in adult females, and 11-16 g/dL in children. The total concentration of oxygen bound to haemoglobin may therefore be proportional to the oxygen saturation and the concentration of haemoglobin in the blood.

The concentration of oxygen dissolved in the plasma may be considered directly proportional to the partial pressure of oxygen in the blood and can be calculated by using a relation referred to as Henry's Law. At body temperature, about 0.0033 mL of oxygen is known to dissolve in 1 mL of blood plasma per mmHg of partial pressure of oxygen.

The above relations can be used to determine the concentration of oxygen, or oxygen content, in the oxygen-rich blood as well as in the oxygen-poor blood. This allows for the oxygen uptake VO₂ in the blood (in terms of, e.g., L/min) to be determined 220 by comparing the concentration of oxygen in the oxygen-rich blood with the concentration of oxygen in the oxygen-poor blood:

$$( Eq . 1 )$$

$${V O}_{2} = {Q}_{blood} \cdot 10 \frac{dL}{L} ( 1.34 \frac{mL}{g} \cdot Hb \cdot \frac{{S}_{a} {O}_{2} - {S}_{v} {O}_{2}}{100} + 0.0 \; 033 \frac{mL}{dL \cdot mmHg} ( {p}_{a} {O}_{2} - {p}_{v} {O}_{2} ) )$$

where Q_{blood} is the flow rate of blood (L/min), Hb is the concentration of haemoglobin (g/dL), SₐO₂ is the saturation of oxygen in the oxygen-rich blood (%), SᵥO₂ is the saturation of oxygen in the oxygen-poor blood (%), pₐO₂ is the partial pressure of oxygen in the oxygen-rich blood (mmHg), and pᵥO₂ is the partial pressure of oxygen in the oxygen-poor blood (mmHg).

As discussed above, the flow rate of blood may be predetermined by operational settings controlling the pump unit 107, whereas the data indicating the concentration of oxygen in the blood may be retrieved from sensor arrangements 122, 123. In the present example, oximeters may be used to measure the concentration of oxygen bound to haemoglobin in the oxygen-rich blood as well as in the oxygen-poor blood, whereas sensors such as optodes or Clark electrodes may be used to measure the concentration of oxygen dissolved in the plasma of the oxygen-rich blood and the oxygen-poor blood, respectively. Each of the first sensor arrangement 122 and the second sensor arrangement 123 may hence comprise a respective oximeter and Clark electrode. The oximeter may be employed to generate sensor data indicative of the oxygen saturations SₐO₂, SᵥO₂, as well as the haemoglobin concentration in the blood, whereas the Clark electrode may be employed to generated sensor data indicating partial pressures pₐO₂, pᵥO₂. As indicated in FIG. 1, the first sensor arrangement 122 may be placed at the outlet of the gas exchange device 110 and the second sensor arrangement 123 placed at the inlet of the gas exchange device 110.

The flow rate of oxygen in the inlet gas may be determined 230 based on the oxygen concentration in the inlet gas and the total flow of the inlet gas. As already mentioned, these parameters may be known from the settings controlling the operation of a gas blender 130. The gas blender 130 may, for example, be configured to deliver a total inlet flow of 4 L/min and an oxygen concentration of 80% or 100% oxygen. It will however be appreciated that the total inlet flow and/or the oxygen concentration in some examples may be determined by one or more sensors, such as a flow meter or a sensor for measuring oxygen concentration.

The flow rate of oxygen in the exhaust gas may be determined 240 based on a difference between the flow rate of oxygen in the inlet gas and the oxygen uptake in the blood. This is based on the realisation that a major part of the oxygen supplied to the gas exchange device 110 is either taken up by the blood or passed as excess oxygen to the exhaust gas flow. This relation can be expressed as:

VO₂=FᵢₙₗₑₜO₂·Qᵢₙₗₑₜ−FₑₓₕₐᵤₛₜO₂·Qₑₓₕₐᵤₛₜ (Eq. 2)

where VO₂ is the oxygen uptake in the blood as determined above, FᵢₙₗₑₜO₂ the concentration of oxygen in the inlet gas, Qᵢₙₗₑₜ the flow rate of the inlet gas, FₑₓₕₐᵤₛₜO₂ the concentration of oxygen in the exhaust gas, and Qₑₓₕₐᵤₛₜ the total flow rate of the exhaust gas. Solving for Qₑₓₕₐᵤₛₜ gives:

$${Q}_{exhaust} = \frac{{F}_{inlet} {O}_{2} \cdot {Q}_{i n l e t} - {V O}_{2}}{{F}_{exhaust} {O}_{2}} \qquad ( Eq . 3 )$$

The oxygen concentration FₑₓₕₐᵤₛₜO₂ in the exhaust gas can be retrieved from sensor data received 250 from a sensor arrangement 124 arranged at the outlet 114 of the gas exchange device 110, as shown in the example of FIG. 1. The oxygen concentration may then be used together with equation 3 above to determine the total flow of the exhaust gas.

The total flow rate of exhaust gas may, in some examples, be used for determining 280 a flow rate of carbon dioxide in the outlet gas. The flow rate of carbon dioxide may be determined based a concentration of carbon dioxide in the exhaust gas and the total flow rate of the exhaust gas. The concentration of carbon dioxide may be received 270 from a carbon dioxide sensor, which may be included in the sensor arrangement 124 at the outlet 114 of the gas exchange device 110. Examples of carbon dioxide sensors include, but are not limited to, infrared sensors correlating the level of absorption of infrared light by carbon dioxide molecules to the concentration of carbon dioxide molecules in the exhaust gas.

The concentration or flow rate of carbon dioxide in the exhaust gas may be used for determining 290 a state of the gas exchange device 110. More specifically, the concentration or flow rate of carbon dioxide may be compared with a reference value, such as a threshold concentration or flow rate. In case the concentration or flow rate of carbon dioxide meets or exceeds the threshold concentration or flow rate, this may indicate that the gas exchange device 110 is well-functioning and hence capable of providing the intended gas-exchange rates. Should the concentration or flow rate of carbon dioxide be lower than the threshold rate, this may indicate that the performance of the membrane 119 has deteriorated and that the gas exchange device 110 may be in need of service or replacement.

It will be appreciated that the term “state” of the gas exchange device 110 generally refers to the condition or performance level of the gas exchange device 110. In a well-functioning or normal state, the gas exchange device 110 may provide a gas exchange rate within acceptance ranges. This state may be determined by comparing the concentration or flow of carbon dioxide in the exhaust gas with a reference measure. In a degraded or malfunction state, the performance of the gas exchange device 110 may have dropped from its normal level. In this state, the membrane 119 may be incapable of facilitating the intended exchange of blood gases. In other examples, the gas exchange device 110 may still be functional, but at risk of not functioning properly. In other words, the patient safety may be compromised. Terms like “degraded”, “impaired”, or “need for service” may be used to describe this state.

The system 100 of the present disclosure may generally comprise one or more processors 140 and one or more non-transitory computer-readable media storing first computer executable instructions that, when executed by the one or more processors, cause the system 100 to perform at least parts of the actions shown in FIG. 2 and described above.

Generally, the system 100 may comprise circuitry which is configured to implement (using one or more non-transitory computer-readable media) the functionality described herein. Suitable processors for the execution of a program of instructions include, by way of example, both general and special purpose microprocessors, and the sole processor or one of multiple processors or cores, of any kind of computer. The processors can be supplemented by, or incorporated in, ASICs (application-specific integrated circuits). Those skilled in the art will understand that the above-described exemplary embodiments may be implemented in any suitable software, hardware, or firmware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above-described method may be embodied as a program containing lines of code stored on a non-transitory computer readable storage medium that, when compiled, may be executed on a processor or microprocessor.

Additionally, variations to the disclosed embodiments can be understood and effected by the skilled person in practising the claimed invention, from a study of the drawing, the disclosure, and the appended claims. Moreover, in the drawings and specification, there have been disclosed preferred examples of the invention and, although specific terms are employed, they are used in a generic and descriptive sense only and not for the purpose of limitation. The scope of the invention is set forth in the following claims, in which the word ‘comprising’ does not exclude other elements or steps, and the indefinite article ‘a’ or ‘an’ does not exclude a plurality.

### CLAUSES

Clause 1. A method (200) for determining a total flow rate of exhaust gas exiting a gas exchange device coupled to a circulatory system of a patient, wherein the gas exchange device is configured to provide oxygen-rich blood to the circulatory system by exposing oxygen-poor blood of the patient to oxygen provided by an inlet gas and to allow passage of excess oxygen to the exhaust gas, the method comprising:
   receiving (210) sensor data indicating an oxygen content in the oxygen-rich blood and an oxygen content in the oxygen-poor blood;
   determining (220) an oxygen uptake in the blood based at least in part on a flow rate of blood through the gas exchange device and a difference between the oxygen content in the oxygen-rich blood and the oxygen content in the oxygen-poor blood;
   determining (230) a flow rate of oxygen in the inlet gas based at least in part on an oxygen concentration in the inlet gas and a total flow rate of the inlet gas;
   determining (240) a flow rate of oxygen in the exhaust gas based at least in part on a difference between the flow rate of oxygen in the inlet gas and the oxygen uptake in the blood;
   receiving (250) sensor data indicating an oxygen concentration in the exhaust gas; and
   determining 260 the total flow rate of the exhaust gas based at least in part on the flow rate of oxygen in the exhaust gas and the oxygen concentration in the exhaust gas.
Clause 2. The method according to clause 1, wherein the sensor data indicating the oxygen content in the oxygen-rich blood and the oxygen content in the oxygen-poor blood are indicative of an amount of oxygen bound to haemoglobin in the oxygen-rich blood and the oxygen-poor blood, respectively, and an amount of oxygen dissolved in the blood plasma of the oxygen-rich blood and the oxygen-poor blood, respectively.
Clause 3. The method according to clause 2, wherein the sensor data indicative of the amount of oxygen dissolved in the blood plasma and of the oxygen-rich blood and the oxygen-poor blood, respectively, is measured by means of a respective blood gas sensor.
Clause 4. The method according to any of the preceding clauses, further comprising:
   reducing a temperature of the exhaust gas; followed by
   exposing a sensor to the exhaust gas, the sensor being configured to generate the sensor data indicating the oxygen concentration in the exhaust gas.
Clause 5. The method according to clause 4, comprising reducing the temperature of the exhaust gas to ambient temperature.
Clause 6. The method according to clause 4 or 5, wherein the sensor is integrated in the gas exchange device.
Clause 7. The method according to any of the preceding clauses, further comprising:
   receiving sensor data (270) indicating a carbon dioxide concentration in the exhaust gas;
   determining (280) a flow rate of carbon dioxide in the outlet gas based at least in part on the carbon dioxide concentration and the total flow rate of the exhaust gas; and
   determining (290), based at least in part on the flow rate of carbon dioxide meets or exceeds a threshold flow rate, a state of the gas exchange device.
Clause 8. The method according to any of the preceding clauses, wherein each of the flow rate of blood, the oxygen concentration in the inlet gas, and the total flow rate of the inlet gas is a predetermined parameter.
Clause 9. A system (100) for exchanging one or more gases with blood in a circulatory system of a patient (10), comprising:
   a gas exchange device (110) comprising a gas inlet (112) for receiving an inlet gas and an outlet (114) for expelling an exhaust gas, wherein the gas exchange device is configured to provide oxygen-rich blood by exposing oxygen-poor blood of the patient to oxygen provided by the inlet gas and to allow passage of excess oxygen to an exhaust gas;
   a sensor arrangement (120) configured to generate sensor data indicating an oxygen content in the oxygen-rich blood, an oxygen content in the oxygen-poor blood, and an oxygen concentration in the exhaust gas;
   one or more processors; and
   one or more non-transitory computer-readable media storing instructions executable by the one or more processors, wherein the instructions, when executed, cause the system to perform operations comprising:
   determining an oxygen uptake in the blood based at least in part on a flow rate of blood through the gas exchange device and a difference between the oxygen content in the oxygen-rich blood and the oxygen content in the oxygen-poor blood;
   determining a flow rate of oxygen in the inlet gas based at least in part on an oxygen concentration in the inlet gas and a total flow rate of the inlet gas;
   determining a flow rate of oxygen in the exhaust gas based at least in part on a difference between the flow rate of oxygen in the inlet gas and the oxygen uptake in the blood; and
   determining the total flow rate of the exhaust gas based at least in part on the flow rate of oxygen in the exhaust gas and the oxygen concentration in the exhaust gas.
Clause 10. The system according to clause 9, wherein the gas exchange device further comprises an emergency outlet (116) through which at least a part of the exhaust gas is expelled.
Clause 11. The system according to clause 9 or 10, wherein the sensor arrangement comprises:
   a first oximeter arranged to measure an oxygen saturation level of the oxygen-rich blood and a second oximeter arranged to measure an oxygen saturation level of the oxygen-poor blood; and
   a first blood gas sensor arranged to measure a partial pressure of oxygen in the oxygen-rich blood and a second blood gas sensor arranged to measure a partial pressure of oxygen in the oxygen-poor blood.
Clause 12. The system according to clause 9 or 10, wherein the operations further comprise at least one of:
   determining an oxygen saturation level of the oxygen-rich blood based on a partial pressure of the oxygen-rich blood; and
   determining an oxygen saturation level of the oxygen-poor blood based on a partial pressure of the oxygen-poor blood.

1. A method for determining a total flow rate of exhaust gas exiting a gas exchange device coupled to a circulatory system of a patient, wherein the gas exchange device is configured to provide oxygen-rich blood to the circulatory system by exposing oxygen-poor blood of the patient to oxygen provided by an inlet gas and to allow passage of excess oxygen to the exhaust gas, the method comprising:
receiving sensor data indicating an oxygen content in the oxygen-rich blood and an oxygen content in the oxygen-poor blood;
determining an oxygen uptake in the blood based at least in part on a flow rate of blood through the gas exchange device and a difference between the oxygen content in the oxygen-rich blood and the oxygen content in the oxygen-poor blood;
determining a flow rate of oxygen in the inlet gas based at least in part on an oxygen concentration in the inlet gas and a total flow rate of the inlet gas;
determining a flow rate of oxygen in the exhaust gas based at least in part on a difference between the flow rate of oxygen in the inlet gas and the oxygen uptake in the blood;
receiving sensor data indicating an oxygen concentration in the exhaust gas; and
determining the total flow rate of the exhaust gas based at least in part on the flow rate of oxygen in the exhaust gas and the oxygen concentration in the exhaust gas.

2. The method according to claim 1, wherein the sensor data indicating the oxygen content in the oxygen-rich blood and the oxygen content in the oxygen-poor blood are indicative of an amount of oxygen bound to haemoglobin in the oxygen-rich blood and the oxygen-poor blood, respectively, and an amount of oxygen dissolved in the blood plasma of the oxygen-rich blood and the oxygen-poor blood, respectively.

3. The method according to claim 2, wherein the sensor data indicative of the amount of oxygen dissolved in the blood plasma of the oxygen-rich blood and the oxygen-poor blood, respectively, is measured by means of a respective blood gas sensor.

4. The method according to claim 1, further comprising:
reducing a temperature of the exhaust gas; followed by
exposing a sensor to the exhaust gas, the sensor being configured to generate the sensor data indicating the oxygen concentration in the exhaust gas.

5. The method according to claim 4, comprising reducing the temperature of the exhaust gas to ambient temperature.

6. The method according to claim 4, wherein the sensor is integrated in the gas exchange device.

7. The method according to claim 1, further comprising:
receiving sensor data indicating a carbon dioxide concentration in the exhaust gas;
determining a flow rate of carbon dioxide in the exhaust gas based at least in part on the carbon dioxide concentration and the total flow rate of the exhaust gas; and
determining, based at least in part on whether the flow rate of carbon dioxide meets or exceeds a threshold flow rate, a state of the gas exchange device.

8. The method according to claim 1, wherein each of the flow rate of blood, the oxygen concentration in the inlet gas, and the total flow rate of the inlet gas is a predetermined parameter.

9. A system for exchanging one or more gases with blood in a circulatory system of a patient, comprising:
a gas exchange device comprising a gas inlet for receiving an inlet gas and an outlet for expelling an exhaust gas, wherein the gas exchange device is configured to provide oxygen-rich blood by exposing oxygen-poor blood of the patient to oxygen provided by the inlet gas and to allow passage of excess oxygen to an exhaust gas;
a sensor arrangement configured to generate sensor data indicating an oxygen content in the oxygen-rich blood, an oxygen content in the oxygen-poor blood, and an oxygen concentration in the exhaust gas;
one or more processors; and
one or more non-transitory computer-readable media storing instructions executable by the one or more processors, wherein the instructions, when executed, cause the system to perform operations comprising:
determining an oxygen uptake in the blood based at least in part on a flow rate of blood through the gas exchange device and a difference between the oxygen content in the oxygen-rich blood and the oxygen content in the oxygen-poor blood;
determining a flow rate of oxygen in the inlet gas based at least in part on an oxygen concentration in the inlet gas and a total flow rate of the inlet gas;
determining a flow rate of oxygen in the exhaust gas based at least in part on a difference between the flow rate of oxygen in the inlet gas and the oxygen uptake in the blood; and
determining the total flow rate of the exhaust gas based at least in part on the flow rate of oxygen in the exhaust gas and the oxygen concentration in the exhaust gas.

10. The system according to claim 9, wherein the gas exchange device further comprises an emergency outlet through which at least a part of the exhaust gas is expelled.

11. The system according to claim 9, wherein the sensor arrangement comprises:
a first oximeter arranged to measure an oxygen saturation level of the oxygen-rich blood and a second oximeter arranged to measure an oxygen saturation level of the oxygen-poor blood; and
a first blood gas sensor arranged to measure a partial pressure of oxygen in the oxygen-rich blood and a second blood gas sensor arranged to measure a partial pressure of oxygen in the oxygen-poor blood.

12. The system according to claim 9, wherein the operations further comprise at least one of:
determining an oxygen saturation level of the oxygen-rich blood based on a partial pressure of the oxygen-rich blood; or
determining an oxygen saturation level of the oxygen-poor blood based on a partial pressure of the oxygen-poor blood.

13. The method according to claim 7, further comprising issuing a warning or signal indicating that the gas exchange device needs to be replaced when the flow rate of the carbon dioxide is lower than the threshold flow rate.

14. The system according to claim 9, wherein the sensor arrangement is further configured to generate sensor data indicating a carbon dioxide concentration in the exhaust gas, and wherein the operations further comprise:
determining a flow rate of carbon dioxide in the exhaust gas based at least in part on the carbon dioxide concentration and the total flow rate of the exhaust gas; and
determining a state of the gas exchange device based at least in part on whether the flow rate of carbon dioxide meets or exceeds a threshold flow rate.

15. The system according to claim 14, wherein the operations further comprise issuing a warning or signal indicating that the gas exchange device needs to be replaced when the flow rate of the carbon dioxide is lower than the threshold flow rate.
